(19) United States
(12) Patent Application Publication
(10) Pub. No.: US 2022/0347495 A1
(43) Pub. Date: Nov. 3, 2022

(54) PAIN RELIEF APPARATUS INDUCED SPIRAL VORTEX BASED ON ULTRASOUND AND VACUUM PULSE

(71) Applicant: Day JOH, Daegu (KR)

(72) Inventor: Day JOH, Daegu (KR)

(21) Appl. No.: 17/406,889

(22) Filed: Aug. 19, 2021

(30) Foreign Application Priority Data
Apr. 30, 2021 (KR) 10-2021-0056220

(51) Int. Cl. A61N 7/00

(52) U.S. Cl. CPC A61N 7/00; A61N 2007/0034; A61N 2007/0073

(57) ABSTRACT

Proposed is an ultrasound and vacuum pulse generator capable of generating a spiral vortex and a pain relief apparatus using the same, which is characterized in that the physiological function of cells adjacent to a target is activated using a spiral vortex-shaped ultrasound.

### CROSS REFERENCE TO RELATED APPLICATION OF THE INVENTION

This application claims priority to and the benefit of Korean patent application No. 10-2021-0056220, filed on Apr. 30, 2021, the disclosure of which is incorporated herein by reference in its entirety.

### BACKGROUND OF THE INVENTION

### Field of the Invention

The present invention relates to an ultrasound and vacuum pulse generator capable of generating a spiral vortex and a pain relief apparatus using the same.

### Background of the Related Art

High-Intensity Focused Ultrasound (HIFU) is used for removing skin wrinkles or in the treatment of cancer.

In the HIFU treatment method, ultrasonic energy is focused within the body so as to heat the tissue to a temperature of approximately 70° C. instantaneously to destroy tissues or cells of the target regions.

In the case of focusing the heat on the Superficial Musculoaponeurotic System (SMAS) layer which exists between layers of muscles and subcutaneous fat, the wrinkles may be removed as collagen degeneration occurs.

In addition, when applying the HIFU to tumors, strong heat is aimed at the tumors where the cavitation and waves in tissues generate, and the tumors may be destroyed.

Conventional HIFU is conducted to focus strong heat on a single target, and there is a risk of inducing thermal injury, and if the ultrasonic energy does not focus precisely on the target, the problems of damaging normal cells or normal tissues may be raised. In addition, the single point method for applying ultrasound energy has a problem in that it is difficult to treat a plurality of targets at once, which leads to a longer time of treatment.

The conventional HIFU also has a problem in that it is difficult to provide treatment for adjacent target regions. In the case of a conventional HIFU is irradiated to the target, the adjacent target region is in a thermal equilibrium state, and the oxygen mobility is made difficult in the adjacent target capillary, therefore a problem may be raised since there is a lack of oxygen.

### SUMMARY OF THE INVENTION

Therefore, the present invention has been made in view of the above problems, and it is an object of the present invention to provide an ultrasound generator capable of treating a target region without destroying thermal injury and adjacent tissues.

In addition, another object of the present invention is to provide an ultrasound generator capable of activating the physiological functions of the capillary of the adjacent target region by destroying the thermal equilibrium of the adjacent target region.

To accomplish the above objects, according to one aspect of the present invention, there is provided an ultrasound generator comprising: a main body having an open lower surface in a trumpet-shape that is hollow inside, a first piezoelectric element attached on a first surface of the main body, a second piezoelectric element attached on the first surface of the main body, and spaced apart from the first piezoelectric element.

At least one of a frequency band and intensity of a voltage applied to an anode of the first piezoelectric element and an anode of the second piezoelectric element may be controlled.

In the ultrasound generator, the frequency band of the ultrasound generated from the first piezoelectric element and the frequency band of the ultrasound generated from the second piezoelectric element may be different, or the amplitude of the ultrasound generated from the first piezoelectric element and the amplitude of the ultrasound generated from the second piezoelectric element may be different.

The first piezoelectric element and the second piezoelectric element may include a piezoelectric portion, a cathode, and an anode, wherein the piezoelectric portion may comprise piezoelectric materials and may include at least one of barium titanate, Rossel salt, crystal, quartz, ceramic, plastic, and graphene.

The cathode of the first piezoelectric element and the cathode of the second piezoelectric element may be configured and coated with a conductive material on the outer surface of the piezoelectric portion, the anode of the first piezoelectric element and the anode of the second piezoelectric element may be configured and coated with the conductive material on the inner surface to the outer surface of the piezoelectric portion, the cathode and the anode of the first piezoelectric element may be spaced apart to each other on the outer surface of the piezoelectric portion, and the cathode and anode of the second piezoelectric element may be spaced apart to each other on the outer surface of the piezoelectric portion.

On the top of the main body, a vacuum generating device is connected and configured to form a sound pressure in the main body, and a vacuum absorption may take place in the main body.

The ultrasound generator may be configured to generate a spiral vortex-shaped ultrasound on which an ultrasound generated in the first piezoelectric element and an ultrasound generated in the second piezoelectric element are superimposed.

A central portion of the spiral vortex-shaped ultrasound may reach a target, and to an adjacent portion of the spiral vortex-shaped ultrasound, therefore, the physiological function of the target adjacent tissues or target adjacent of the unit cell may be activated.

A pain relief apparatus according to an embodiment of the present invention may include at least one ultrasound generator aforementioned.

### BRIEF DESCRIPTION OF THE DRAWINGS

FIG. 1 is a view showing an ultrasound generator and a vacuum generating device being connected.

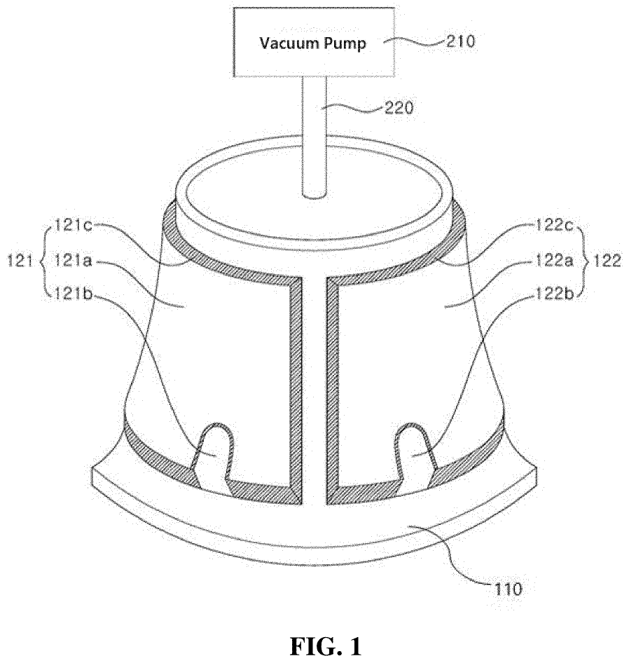

FIG. 2 is a view showing a piezoelectric element according to an embodiment of the present invention.

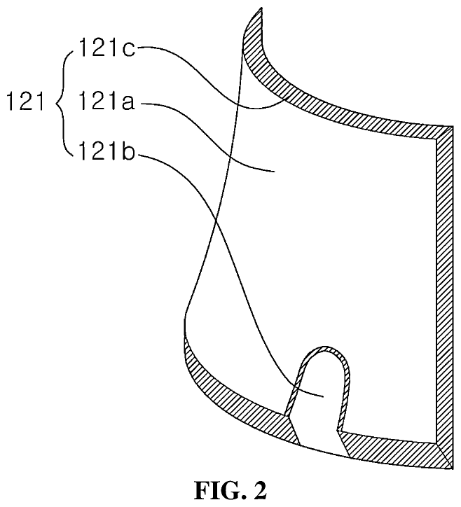

FIG. 3 is a view showing a spiral vortex-shaped ultrasound of the present invention.

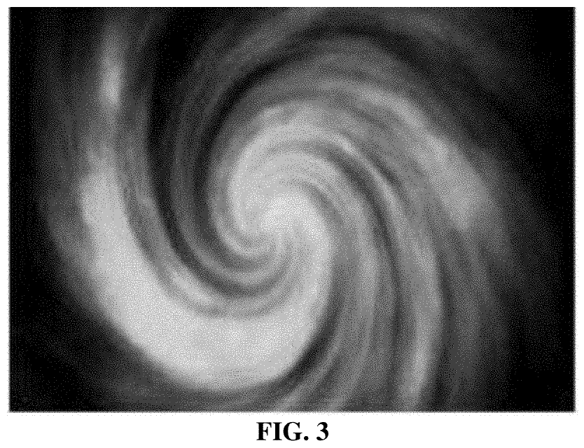

### DETAILED DESCRIPTION OF THE PREFERRED EMBODIMENT

### Ultrasound Generator

An ultrasound generator according to an embodiment of the present invention may comprise a main body 110 and a plurality of piezoelectric portions.

### 1. Main Body 110

A main body 110 according to an embodiment of the present invention may be configured in a trumpet shape that is hollow inside. The main body 110 is configured to have an open lower surface, and a lower cross-sectional surface of the main body 110 is formed in a donut shape. Both upper and lower cross-sectional surfaces of the main body are in a circular shape, and the upper surface of the main body has a narrower cross-sectional surface compared to the lower surface of the main body.

### 2. Piezoelectric Element

An ultrasound generator of the present invention may comprise a plurality of piezoelectric elements 121 and 122. The piezoelectric elements may be configured in plural on the outer surface of the main body 110.

The ultrasound generator may include a first piezoelectric element 121 and a second piezoelectric element 122. Both the first piezoelectric element 121 and the second piezoelectric element 122 are attached and spaced apart at a predetermined distance on the trumpet-shaped main body 110.

Both the first piezoelectric element 121 and the second piezoelectric element 122 are configured to have a predetermined curvature on both sides and spaced apart downwards so as to be attached to the trumpet-shaped main body 110. The first piezoelectric element 121 and the second piezoelectric element 122 may have an equal shape and cross-sectional area.

The first piezoelectric element 121 and the second piezoelectric element 122 may equally include anodes 121b and 122b, piezoelectric portions 121c and 122c, and cathodes 121a and 122a.

The piezoelectric portion 121c of the first piezoelectric element may comprise piezoelectric materials. The piezoelectric materials include at least one of barium titanate, Rossel salt, crystal, quartz, ceramic, plastic, and graphene.

The cathode 121a of the first piezoelectric element may be configured by coating with conductive materials on the outer surface of the piezoelectric portion. The anode 121b of the first piezoelectric element is configured extendedly from the inner surface of the piezoelectric portion 121c to the outer surface of the piezoelectric portion 121c. The cathode 121a and anode 121b of the first piezoelectric element may be coated with the same materials.

The cathode 121a of the first piezoelectric element and the anode 121b of the first piezoelectric element may be arranged to be spaced apart at a predetermined distance on the outer surface of the piezoelectric portion 121c. At this time, the conductive material is not coated between the cathode 121a of the first piezoelectric element and the anode 121a of the first piezoelectric element, so that the piezoelectric material of the piezoelectric portion 121c may serve as an insulator between cathode 121a and the anode 121b.

The piezoelectric portion 122c of the first piezoelectric element may be comprised of piezoelectric materials. The piezoelectric materials include at least one of barium titanate, Rossel salt, crystal, quartz, ceramic, plastic, and graphene.

The cathode 122a of the first piezoelectric element may be configured by coating with conductive materials on the outer surface of the piezoelectric portion. The anode 122b of the first piezoelectric element is configured extendedly from the inner surface of the piezoelectric portion to the outer surface of the piezoelectric portion. The cathode 122a and anode 122b of the first piezoelectric element may be coated with the same materials.

The cathode 122a of the second piezoelectric element and the anode 122b of the first piezoelectric element may be arranged to be spaced apart at a predetermined distance on the outer surface of the piezoelectric portion 122c. At this time, the conductive material is not coated between the cathode 122a of the second piezoelectric element and the anode 121a of the first piezoelectric element, so that the piezoelectric material of the piezoelectric portion 122c may serve as an insulator between cathode 122a and the anode 122b.

To allow the frequency band generated from the first piezoelectric element 121 and the frequency band generated from the second piezoelectric element 122 to be different, the frequency band and/or the voltage applied to the cathode 121a of the first piezoelectric element 121 and the cathode 122a of the second element may be controlled.

To allow the frequency band generated from the first piezoelectric element 121 and the frequency amplitude generated from the second piezoelectric element 122 to be different, the frequency amplitude and/or the voltage applied to the cathode 121a of the first piezoelectric element 121 and the cathode 122a of the second element may be controlled.

Although two piezoelectric elements 121 and 122 may be attached on the outer surface of the main body 110 as described above, within the present embodiment, two piezoelectric elements, is not limited to but may include more than two piezoelectric elements may be included, if needed, without departing from the scope and spirit of the present invention.

For example, the ultrasound generator may include a first piezoelectric element 121, a second piezoelectric element 122, and a third piezoelectric element (not shown). The first piezoelectric element 121, the second piezoelectric element 122, and the third piezoelectric element may be divided into equal three parts with respect to the center of the main body 110. The first piezoelectric element 121 to the third piezoelectric element (not shown) may be designed to have an equal shape and surface area. The frequencies generated from the first piezoelectric element 121 to the third piezoelectric element may control the frequency amplitude and/or the voltage applied to the cathode 121a of the first piezoelectric element 121 and the cathode 122a of the second element to allow the frequency band and/or amplitude to be different.

For example, voltages of 60V, 70V, and 80V may be applied to the first piezoelectric element 121, the second piezoelectric element 122, and the third piezoelectric element respectively.

When the intensity and/or frequency band of a plurality of voltage applied is controlled to be different, the frequency band and/or amplitude of frequency generated from a plurality of piezoelectric elements may be different. If a plurality of ultrasound generated in such a manner is irradiated on a deep target, the plurality of ultrasound having different frequency bands and amplitude may be superimposed to each other and interference may occur. Therefore, when the ultrasound having different frequency bands and amplitude superimpose to each other, the energy equilibrium may be destroyed and the spiral vortex-shaped ultrasound may be generated.

### 3. Vacuum Generating Device

According to an embodiment of the present invention, a vacuum generating device 210 may be connected to the top surface of the main body 110. Preferably, a vacuum pump 210 may be arranged on the outside of the main body 110. A conduit is connected between the main body 110 and the vacuum pump, thereby the vacuum pulse generated when operating the vacuum pump 210 is transferred to a hollow of the main body 110 via the conduit 220, and the sound pressure is exerted in the hollow of the main body 110. Through the sound pressure, a skin region that corresponds to the target is made to be absorbed in the hollow of the main body 110.

Further, when the ultrasound is generated from the plurality of the piezoelectric elements in the state of sound pressure being exerted on the skin, even stronger spiral vortex-shaped ultrasound may be generated as the ultrasonic energy is scattered.

In the case of the spiral vortex-shaped ultrasound is irradiated on the target, the center portion of the spiral vortex may reach the target, and the adjacent spiral vortex may be reached to the adjacent target portion. In the case of the spiral vortex-shaped ultrasound is irradiated on the target, ultrasound cavitation may take place on the target adjacent portion, and therefore nano-unit-sized fine bubbles may be generated in the adjacent portion of the target. That is, when the spiral vortex-shaped ultrasound is applied to the target, nano-unit-sized fine bubbles may be generated in the capillary of the target adjacent portion. The fine bubbles generated in such a manner may activate the physiological function of the capillary.

In addition, as for the spiral vortex-shaped ultrasound, the ultrasound having lower temperature compared to the High-Intensity Focused Ultrasound (HIFU) is reached to the target, thereby there is no risk of suffering a thermal injury.

### Pain Relief Apparatus

A pain relief apparatus according to an embodiment of the present invention may include at least one ultrasound generator aforementioned. The pain relief apparatus may preferably include a plurality of the ultrasound generator aforementioned. Even stronger spiral vortex-shaped ultrasound may be generated by inclining the plurality of the ultrasound generator in different inclinations respectively.

The pain relief apparatus may be used for inflammatory pain or cancerous pain.

The present invention is advantageous in that a thermal injury of skin and a target of adjacent tissues may be treated without any damages by attaching a plurality of piezoelectric elements on a trumpet-shaped main body, controlling a frequency and an intensity of a voltage applied to the plurality of piezoelectric elements, and using an ultrasound generator that generates a spiral vortex-shaped ultrasound.

What is claimed is:

1. An ultrasound generator comprising:
a main body having an open lower surface in a trumpet shape that is hollow inside;
a first piezoelectric element attached on a first surface of the main body;
a second piezoelectric element attached on the first surface of the main body, and spaced apart from the first piezoelectric element; and
controlling at least one of a frequency band and intensity of a voltage applied to an anode of the first piezoelectric element and an anode of the second piezoelectric element.

2. The ultrasound generator according to claim 1, wherein
the ultrasound generator configured to comprise:
a frequency band of the ultrasound generated from the first piezoelectric element and the frequency band of the ultrasound generated from the second piezoelectric element is different; and
an amplitude of the ultrasound generated from the first piezoelectric element and the amplitude of the ultrasound generated from the second piezoelectric element are different.

3. The ultrasound generator according to claim 1, wherein
the first piezoelectric element and the second piezoelectric element include a piezoelectric portion, a cathode, and an anode; and
the piezoelectric portion is formed of piezoelectric materials, wherein the piezoelectric materials include at least one of barium titanate, Rossel salt, crystal, quartz, ceramic, plastic, and graphene.

4. The ultrasound generator according to claim 3, wherein
a cathode of the first piezoelectric element and a cathode of the second piezoelectric element configured by coating with conductive materials on the outer surface of the piezoelectric portion;
an anode of the first piezoelectric element and an anode of the second piezoelectric element configured and coated with the conductive material on the inner surface to the outer surface of the piezoelectric portion;
the cathode and the anode of the first piezoelectric element is spaced apart from each other on the outer surface of the piezoelectric portion; and
the cathode and anode of the second piezoelectric element are spaced apart from each other on the outer surface of the piezoelectric portion.

5. The ultrasound generator according to claim 1, wherein
a vacuum generating device is connected and configured to form a sound pressure in the main body on the top of the main body; and
a vacuum absorption is configured to take place in the main body.

6. The ultrasound generator according to claim 2, wherein
the ultrasound generator configured to comprise:
generating a spiral vortex-shaped ultrasound on which an ultrasound generated in the first piezoelectric element and an ultrasound generated in the second piezoelectric element are superimposed.

7. The ultrasound generator according to claim 6, wherein
a central portion of the spiral vortex-shaped ultrasound is configured to reach a target and to an adjacent portion of the spiral vortex-shaped ultrasound; and
activating the physiological function of the target adjacent tissues or target adjacent of the unit cell.

8. A pain relief apparatus comprising at least the ultrasound generator of claim 1.
